(19) United States
(12) Patent Application Publication
(10) Pub. No.: US 2006/0183890 A1
(43) Pub. Date: Aug. 17, 2006

(54) Isopropanolate of azithromycin and method of manufacturing

(76) Inventors: Tim Fat Tam, Woodbridge (CA); Khashayar Karimian, Toronto (CA); Shui Sheng Hu, Ontario (CA); Anna Chow, Ontario (CA); Richard William Storey, Ontario (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(21) Appl. No.: 10/550,104

(22) PCT Filed: Mar. 19, 2004
(86) PCT No.: PCT/CA04/00406

§ 371 Date: Sep. 21, 2005

(30) Foreign Application Priority Data
Mar. 21, 2003 (CA) 2422972

(51) Int. Cl. A61K 31/7052; C07H 17/08

(52) U.S. Cl. 536/7.4

(57) ABSTRACT

Azithromycin isopropanolate of the empirical formula azithromycin:[H₂0]ₓ:[isopropanol]_{y} is obtained from the crystallization of azithromycin in isopropanol and water. The x and y values is confirmed by single X-ray diffraction determination. In one embodiment x=1.5 and y=0.25. In another embodiment x=0.75 and y=0.5.

### BACKGROUND OF THE INVENTION

Azithromycin, 9-Deoxo-9a-aza-9a-methyl-9a-homoerythromycin A, is a semi-synthetic macrolide antibiotic (U.S. Pat. No. 4,517,359), which can be classified as a member of the second-generation erythromycin antibacterial agent. Azithromycin has the following structure (I):

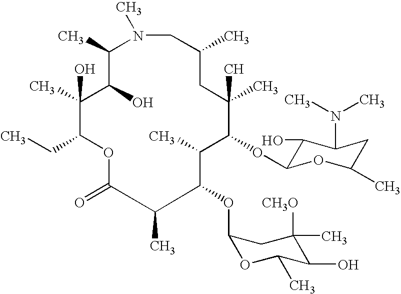

A useful crystal form of azithromycin intended for pharmaceutical use must be free of toxic organic solvent such as tetrahydrofuran and chloroform. The commonly known azithromycin crystal forms are azithromycin monohydrate and azithromycin dihydrate.

According to Canadian Patent 1,191,843, anhydrous azithromycin can be obtained by evaporating a chloroform solution of the material to give a foam. The residual solvent is difficult to remove and the non-crystalline material cannot be easily purified. This material is unsuitable for pharmaceutical use.

EP 941,999 reports a method for the preparation of azithromycin monohydrate and dihydrate from acetone/basic water crystallization. U.S. Pat. No. 6,245,903 reports a crystalline form azithromycin isopropanol clathrate with a proposed ratio of azithromycin:water:isopropanol of 1:1:0.3, and a process for the preparation of the same from solid azithromycin by adding water to a solution of azithromycin in isopropanol. WO2094843 reports a method for the preparation of azithromycin Form M from isopropanol/water and the suggested ratio of azithromycin:water:isopropanol is 1:1:0.5. Form M is prepared by adding cold water to a solution of azithromycin in isopropanol.

WO 03/077830 reports a process for the preparation of [azithromycin].[H₂0] from [azithromycin].[H₂0]ₓ.[S]_{y} wherein S is an organic solvent, which is at least partially miscible with water. The value x is restricted to 1, 1.25, 1.5 or 2 and y is restricted to y is 0. 0.5 and 1. There is no procedure shown in WO 03/077830 for the preparation of [azithromycin].[H₂0]ₓ.[S]_{y} with the above x and y values from isopropanol and water. Example 2 of WO 03/077830 reports the preparation of [azithromycin].[H₂0]_{1.5}.[isopropanol]_{0.5} from isopropanol and sodium hydroxide solution at pH 9.8. [Azithromycin].[H₂0]_{1.5}.[isopropanol]_{0.5} has a formula weight of 806 and a theoretical isopropanol content of 3.72%. Form M ([azithromycin].[H₂0]₁.[isopropanol]_{0.5}) has a formula weight of 797 and a theoretical isopropanol content of 3.76%. Both forms of azithromycin have theoretical isopropanol content in excess of 3.6%. In addition to the isopropanol within the molecular formula, the crystalline solid may also contain surface isopropanol resulting in even a higher percentage of isopropanol in these substances.

The crystalline azithromycin.(H₂O)ₓ.[isopropanol]_{y} of this invention differs in empirical formula from the azithromycin reported in the literature. The value of x is not 1 and therefore the material is not an isopropanolate solvate form of azithromycin monohydrate. Azithromycin isopropanolate of formula azithromycin.(H₂O)ₓ.[isopropanol]y wherein x=1.5 and y=0.25 or x=0.75 and y=0.5 is unknown in the literature and the form azithromycin.(H₂O)_{1.5}.[isopropanol]_{0.25} has a formula weight of 791 and a theoretical % isopropanol of 1.9% which is lower than the % isopropanol content of all the other forms known in the literature. They are prepared from non-crystalline azithromycin. Non-crystalline azithromycin can be prepared by extracting a solution of azithromycin in dilute acetic acid with ethyl acetate to remove any non-basic drug related substances. The acetic acid fraction is neutralized with base, and then the azithromycin is extracted into ethyl acetate. The ethyl acetate fraction is dried and the solvent is evaporated under vacuo to give an oil. The oil is co-evaporated with isopropanol three times before it is crystallized from isopropanol and water. The solid is crystallized from isopropanol and water. The solid is filtered, and dried under vacuo at 45 to 55° C. for 12 to 16 hrs. When the solid is dissolved in isopropanol at 20 to 30° C. and crystallized with the addition of water, azithromycin.(H₂O)ₓ.[isopropanol]_{y} with the above x and y values are obtained (depending on the amount of water added). The ratio of x and y is controlled by the amount of water added. When the solvent ratio of isopropanol to water is in the order of [1-2] to 1 by volume, the crystalline form obtained is azithromycin (H₂O)ₓ.[isopropanol]y with x=1.5 and y=0.25. The structure and the empirical formula of this new solvate have been determined by single crystal x-ray diffraction determination. When a minimum amount of water is added to a saturated solution of azithromycin in isopropanol, azithromycin.(H₂O)ₓ.[isopropanol]y (for example in the order of 1:4 water:isopropanol) crystal with x=0.75 and y=0.5 is obtained. The crystal structure and the empirical formula of this new solvate are determined by single crystal x-ray diffraction determination.

For the azithromycin.(H₂O)ₓ.[isopropanol]_{y} with different ratio of, the solvent molecules produced, the unique crystalline lattice is maintained. The PXD pattern and the FT-IR spectrum of these two different azithromycin.(H₂O)ₓ.[isopropanol]_{y} wherein x=1.5 and y=0.25 and x=0.75 and y=0.25 are the same. Their unit cell values and other crystallographic data are presented in Table 1 and FIG. 1.

### DESCRIPTION OF ASPECTS OF THE INVENTION

According to one aspect of the invention a crystalline form of azithromycin isopropanolate with the formula azithromycin.(H₂O)ₓ.[isopropanol]_{y} wherein x=0.75 and y 0.5 is provided, characterized by single crystal structure results summarized in Table 1, the similar powder X-ray diffraction pattern and FT-IR spectrum in FIG. 2 and FIG. 3, respectively.

According to another aspect of the invention a crystalline form of azithromycin isopropanolate with the formula azithromycin.(H₂O)ₓ.[isopropanol]_{y} wherein x=1.5 and y=0.25 is provided, characterized by single crystal structure results summarized in Table 1, the powder X-ray diffraction pattern in FIG. 4 and the FT-IR spectrum shown in FIG. 5.

According to another aspect of the invention this invention relates to processes for the preparation of azithromycin.(H₂O)_{1.5}.[isopropanol]_{0.25} and azithromycin.(H₂O)_{0.75}.[isopropanol]_{0.5}. The form obtained depends on the ratio of water to isopropanol used in the crystallization as discussed above.

A process may comprise the following steps:

Preparation of azithromycin:(H₂O)ₓ:[isopropanol]_{y} from non-crystalline azithromycin:
(a) Dissolving solid azithromycin in an acetic acid solution and extracting the solution with ethyl acetate.
(b) The aqueous solution from step (a) is basified with sodium hydroxide solution.
(c) The basic solution from step (b) is extracted with ethyl acetate.
(d) The ethyl acetate solution from step (c) is dried with sodium sulfate. The drying agent is filtered and the filtrate evaporated under vacuo to give non-crystalline azithromycin as a syrup.
(e) The material from step (d) is co-evaporated with isopropanol three times to give a syrup.
(f) The material from step (e) is mixed with isopropanol.
(g) Water is added to the material from step (f).
(h) The insoluble material from step (g) is filtered and dried under vacuo.
(i) The material from step (h) is dissolved in isopropanol. Water is added preferably in the ratios discussed hereafter.
(l) The insoluble material from step (i) is filtered.

Azithromycin.(H₂O)_{X}.[isopropanol]_{y} wherein x=0.75 and y=0.5 is obtained when water is added to a saturated solution of material from step (h) in isopropanol. An example of the ratio of water:isopropanol is in the order of 1:4 (0.25:1).

Azithromycin.(H₂O)ₓ.[isopropanol]y wherein x=1.5 and y=0.25 is obtained when the ratio of water to isopropanol in step (i) is of the order of 1 to [1 to 2].

The crystalline azithromycin.(H₂O)_{1.5}.[isopropanol]_{0.25} and azithromycin.(H₂O)_{0.75}.[isopropanol]_{0.50} of this invention are unknown in the literature and manufactured by a process superior to other reported procedures of isopropanol/water or basic water crystallization. First, these forms of azithromycin may be prepared in high purity and pharmaceutically acceptable quality, because any non-basic drug related substances are removed in the acetic add/ethyl acetate extraction step of the process. Second, purified non-crystalline azithromycin is used as a starting material and can be prepared from any crude solid azithromycin. Third, the use of acidic or basic water is not required for the isopropanol and water crystallization step. Fourth, extensive heating and cooling conditions are not required.

For pharmaceutical use, azithromycin.(H₂O)_{1.5}.[isopropanol]_{0.25} contains a significantly lower theoretical isopropanol content than all other isopropanol pseudopolymorphs of azithromycin. This form of azithromycin provides improved stability and ease of manufacture and use.

### BRIEF DESCRIPTION OF THE DRAWINGS

The following figures illustrate preferred and alternative embodiments of the invention, wherein:

FIG. 1 Stereo-structure of azithromycin:(H₂O)_{0.75}:[isopropanol]_{0.5} (a) verses azithromycin:(H₂O)_{1.5}:[isopropanol]_{0.25} (b)

FIG. 2 Powder X-ray diffraction pattern of azithromycin.(H₂O)_{0.75}.[isopropanol]_{0.5}.

FIG. 3 Single crystal microscope FT-IR spectrum of azithromycin.(H₂O)_{0.75}.[isopropanol]_{0.5}.

FIG. 4 Powder X-ray diffraction pattern of azithromycin.(H₂O)_{1.5}.[isopropanol]_{0.25}.

FIG. 5 Single crystal microscope FT-IR spectrum of azithromycin.(H₂O)_{1.5}.[isopropanol]_{0.25}.

Table 1 Azithromycin:(H₂O)ₓ:[isopropanol]_{y} Single Crystal Structure Information.

### EXAMPLE 1

Preparation of Azithromycin:(H₂O)ₓ:[isopropanol]_{y}

A. Purification of Azithromycin Via Acid/Base Extraction

Azithromycin monohydrate (100 g) was mixed with water (500 ml) in a 2-litre beaker. Acetic acid (17 ml) was added. The mixture was stirred for 15 mins. Ethyl acetate (270 ml) was added. The mixture was stirred for 15 minutes and extracted in a separation funnel. The lower water layer was transferred to 2-litre beaker. Water (100 ml) was added to the ethyl acetate layer and the mixture was extracted. The lower water layer was combined with the aqueous layer from the previous separation. Ethyl acetate (360 ml) was added to the combined aqueous layer, followed by 6N NaOH solution (54 ml). The mixture was stirred for 15 mins, extracted, and then separated. The lower water layer was removed and extracted twice with ethyl acetate (90 ml). The combined ethyl acetate layer was washed with water (100 ml), and the water layer removed. The ethyl acetate solution is dried over sodium sulfate and evaporated to give a foamy material. The foamy material was mixed with isopropanol (86 ml) and evaporated to dryness under reduced pressure at 40° C. This step was repeated twice. The foamy material was mixed with isopropanol (258 ml) to give an approximate total volume of 400 ml in a 600-ml beaker. Water (460 ml) was added slowly with stirring. The insoluble solid was filtered after 2 hrs and dried at 50° C. under vacuo for 16 hrs.

B. Preparation of Azithromycin:(H2O)ₓ:[isopropanol]_{y} Wherein x=1.5, y=0.25.

The material (5 g) from example 1A was dissolved in isopropanol (20 ml) and stirred for 15 mins. Water (10 ml) was added dropwise with stirring. When the addition of water was completed, the stirring bar was removed and the material was allowed to sit for 44 hours. The crystals was filtered and used immediately for single crystal structural determination.

C. Preparation of Azithromycin:(H2O)ₓ:[isopropanol]_{y} Wherein x=1.5, y=0.25.

The material (5 g) from example 1A was dissolved in isopropanol (20 ml) and stirred for 15 mins. Water (20 ml) was added dropwise with stirring. When the addition of water was completed, the stirring bar was removed and the material was allowed to sit for 44 hours. The crystals was filtered and used immediately for single crystal structural determination.

D. Preparation of Azithromycin:(H2O)ₓ:[isopropanol]_{y} Wherein x=0.75, y=0.5

Water (0.1 ml) was added dropwise with stirring to a saturated solution of the material from example 1A (0.5 ml). Crystals were formed after 8 hours. The crystals was filtered and used immediately for single crystal x-ray diffraction structural determination.

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and, not in a limiting sense.

1. A crystalline form of azithromycin.(H₂O)ₓ.[isopropanol]_{y} wherein x and y are selected from
(i) x=0.75 and y=0.5, and
(ii) x=1.5 and y=0.25.

2. Crystalline Azithromycin Isopropanolate of claim 1 wherein x=1.5 and y=0.25.

3. Crystalline Azithromycin Isopropanolate of claim 1 wherein x=0.75 and y=0.5.

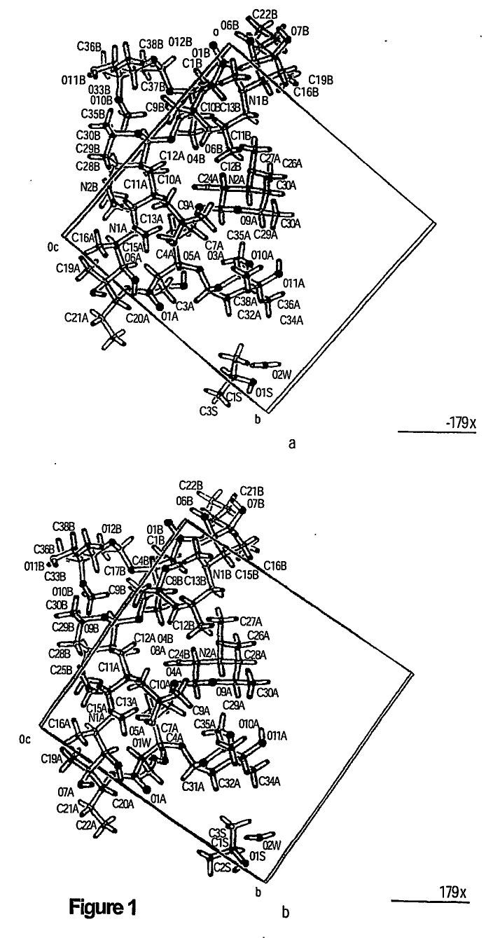

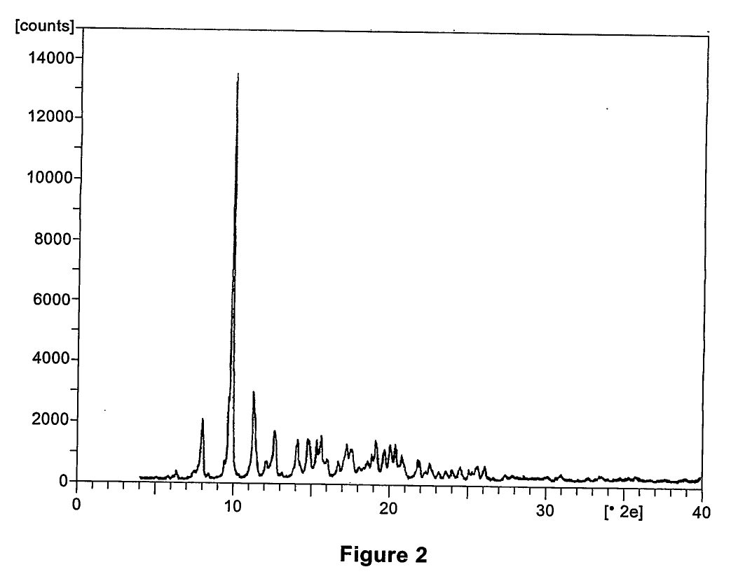

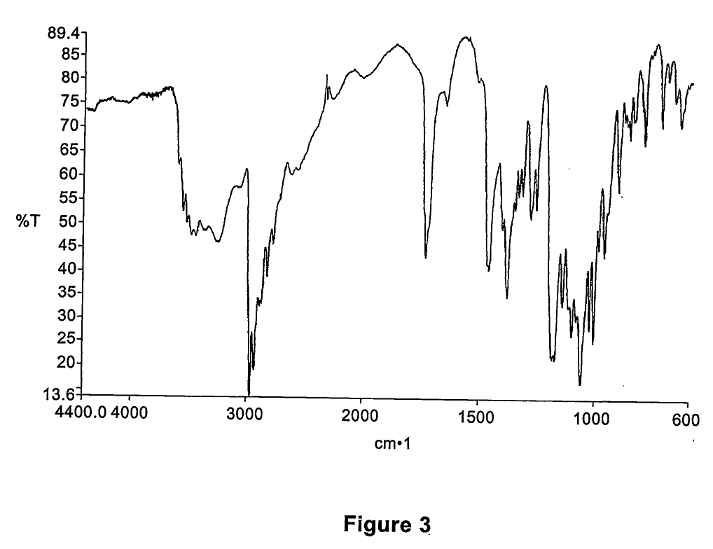

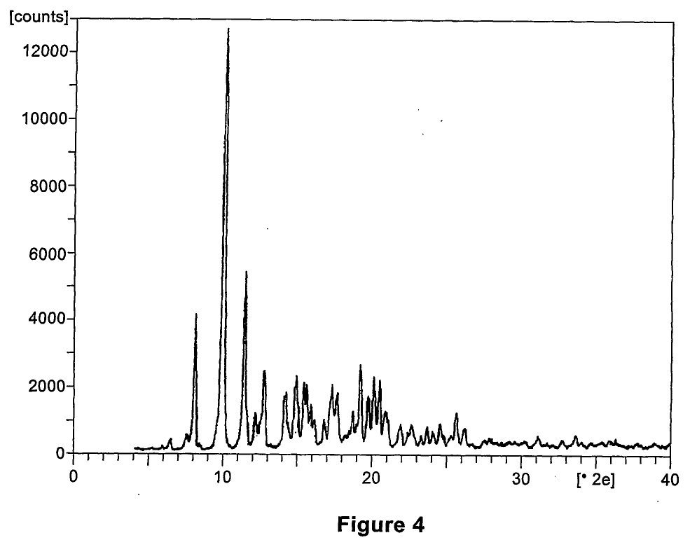

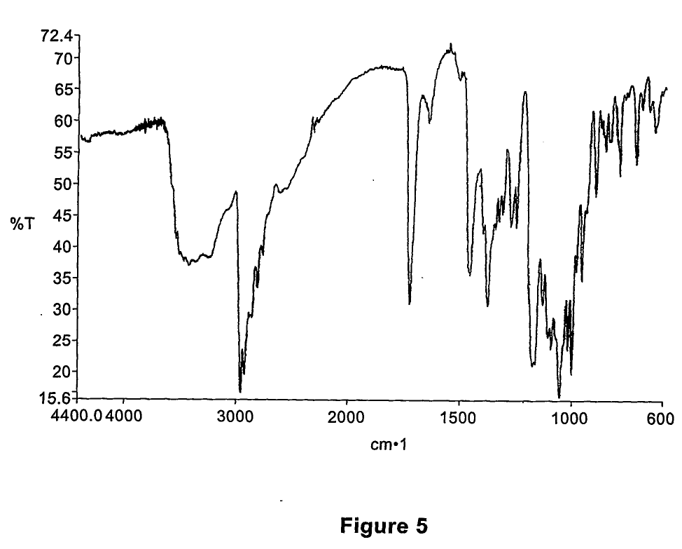

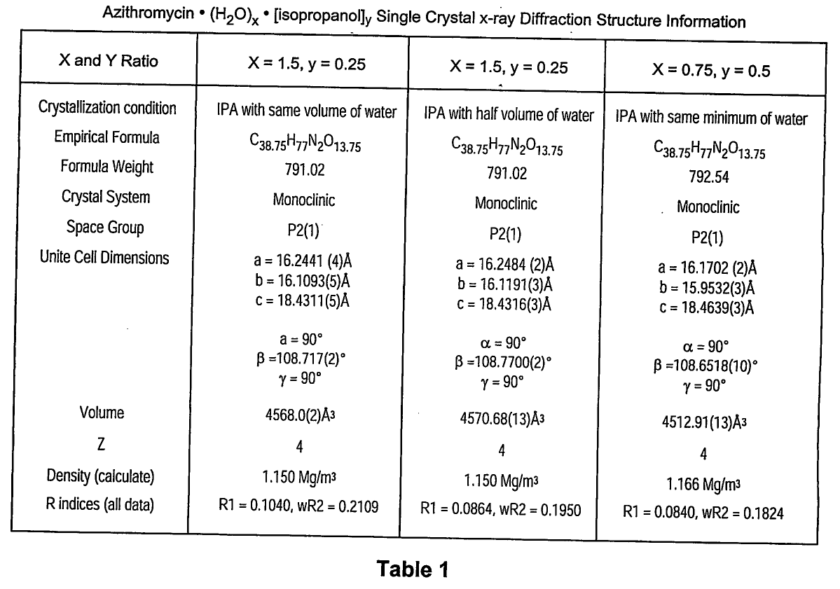

4. The crystalline form of Azithromycin.(H₂O)ₓ.[isopropanol]_{y} having the single crystal structure of FIG. 1(a) wherein x=0.75 and y=0.5.

5. The crystalline form of Azithromycin.(H₂O)ₓ.[isopropanol]_{y} having the single crystal structure of FIG. 1(b) wherein x=1.5 and y=0.25.

6. A process for the preparation of the azithromycin.(H₂O)ₓ.[isopropanol]_{y} wherein x and y are selected from
(i) x=0.75 and y=0.5, and
(ii) x=1.5 and y=0.25
which process comprises the following steps:
(a) dissolving solid azithromycin in an acetic acid solution and extracting the solution with ethyl acetate;
(b) basifying the aqueous solution from step (a) with a sodium hydroxide solution;
(c) extracting the basic solution from step (b) with ethyl acetate;
(d) drying the ethyl acetate solution from step (c) with sodium sulfate, filtering the drying agent and evaporating the filtrate under vacuo to give non-crystalline azithromycin as a syrup;
(e) co-evaporating the material from step (d) with isopropanol three times to give a syrup;
(f) mixing the material from step (e) with isopropanol;
(g) adding water to the material from step (f);
(h) filtering the insoluble material from step (g) and drying under vacuo;
(i) dissolving the material from step (h) in isopropanol and adding water in the ratio of either:
(ia) isopropanol to water in the order of (1-2):1 where x=1.5 and y=0.25, or
(ib) in the ratio of isopropanol to water in the order of 4:1 where x=0.75 and y=0.5;
(j) filtering the insoluble material from step (i).

7. A process for the preparation of the azithromycin.(H₂O)ₓ.[isopropanol]_{y} of claim 2 wherein x=1.5 and y=0.25 which comprises the following steps:
(a) dissolving solid azithromycin in an acetic acid solution and extracting the solution with ethyl acetate;
(b) basifying the aqueous solution from step (a) with a sodium hydroxide solution;
(c) extracting the basic solution from step (b) with ethyl acetate;
(d) drying the ethyl acetate solution from step (c) with sodium sulfate, filtering the drying agent and evaporating the filtrate under vacuo to give non-crystalline azithromycin as a syrup;
(e) co-evaporating the material from step (d) with isopropanol three times to give a syrup;
(f) mixing the material from step (e) with isopropanol;
(g) adding water to the material from step (f);
(h) filtering the insoluble material from step (g) and drying under vacuo;
(i) dissolving the material from step (h) in isopropanol and adding water wherein the ratio of isopropanol to water is in the order of (1-2):1;
(j) filtering the insoluble material from step (i).

8. A process for the preparation of the azithromycin.(H₂O)ₓ.[isopropanol]_{y} of claim 3 wherein x=0.75 and y=0.5 which comprises the following steps:
(a) dissolving solid azithromycin in an acetic acid solution and extracting the solution with ethyl acetate;
(b) basifying the aqueous solution from step (a) with a sodium hydroxide solution;
(c) extracting the basic solution from step (b) with ethyl acetate;
(d) drying the ethyl acetate solution from step (c) with sodium sulfate, filtering the drying agent and evaporating the filtrate under vacuo to give non-crystalline azithromycin as a syrup;
(e) co-evaporating the material from step (d) with isopropanol three times to give a syrup;
(f) mixing the material from step (e) with isopropanol;
(g) adding water to the material from step (f);
(h) filtering the insoluble material from step (g) and drying under vacuo;
(i) dissolving the material from step (h) in isopropanol and adding water wherein the ratio of isopropanol to water is in the order of 4:1;
(j) filtering the insoluble material from step (i).

9. A process for the preparation of the azithromycin.(H₂O)ₓ.[isopropanol]_{y} of claim 4 wherein x=0.75 and y=0.5 which comprises the following steps:
(a) dissolving solid azithromycin in an acetic acid solution and extracting the solution with ethyl acetate;
(b) basifying the aqueous solution from step (a) with a sodium hydroxide solution;
(c) extracting the basic solution from step (b) with ethyl acetate;
(d) drying the ethyl acetate solution from step (c) with sodium sulfate, filtering the drying agent and evaporating the filtrate under vacuo to give non-crystalline azithromycin as a syrup;
(e) co-evaporating the material from step (d) with isopropanol three times to give a syrup;
(f) mixing the material from step (e) with isopropanol;
(g) adding water to the material from step (f);
(h) filtering the insoluble material from step (g) and drying under vacuo;
(i) dissolving the material from step (h) in isopropanol and adding water wherein the ratio of isopropanol to water is in the order of 4:1;
(j) filtering the insoluble material from step (i).

10. A process for the preparation of the azithromycin.(H₂O)ₓ.[isopropanol]_{y} of claim 5 wherein x=1.5 and y=0.25 which comprises the following steps:
(a) dissolving solid azithromycin in an acetic acid solution and extracting the solution with ethyl acetate;
(b) basifying the aqueous solution from step (a) with a sodium hydroxide solution;
(c) extracting the basic solution from step (b) with ethyl acetate;
(d) drying the ethyl acetate solution from step (c) with sodium sulfate, filtering the drying agent and evaporating the filtrate under vacuo to give non-crystalline azithromycin as a syrup;
(e) co-evaporating the material from step (d) with isopropanol three times to give a syrup;
(f) mixing the material from step (e) with isopropanol;
(g) adding water to the material from step (f);
(h) filtering the insoluble material from step (g) and drying under vacuo;
(i) dissolving the material from step (h) in isopropanol and adding water wherein the ratio of isopropanol to water is in the order of (1-2):1;
(j) filtering the insoluble material from step (i).

11. A crystalline form of azithromycin.(H₂O)ₓ.[isopropanol]_{y} wherein x=0.75 and y=0.5 made by the process of claim 6, 8 or 9.

12. A crystalline form of azithromycin.(H₂O)ₓ.[isopropanol]_{y} wherein x=1.5 and y=0.25 made by the process of claim 6, 7 or 10.

13. A crystalline form of azithromycin.(H₂O)_{0.75}.[isopropanol]_{0.5} having the single crystal structure of FIG. 1(a) made by the process of claim 9.

14. A crystalline form of azithromycin.(H₂O)_{0.75}.[isopropanol]_{0.5} having the single crystal structure of FIG. 1(b) made by the process of claim 10.

15. A crystalline form of azithromycin.(H₂O)_{0.75}.[isopropanol]_{0.5} having the single crystal structure of FIG. 1(a).

16. A crystalline form of azithromycin.(H₂O)_{0.75}.[isopropanol]_{0.5} having the single crystal structure of FIG. 1(b).
